(19) United States
(12) Patent Application Publication
(10) Pub. No.: US 2026/0207788 A1
(43) Pub. Date: Jul. 23, 2026

(54) HEAVY ISOTOPE 3-BP MOLECULES, COMPOSITIONS, AND TREATMENTS

(71) Applicant: KODISCOVERY, LLC, Baltimore, MD (US)

(72) Inventor: Young Hee Ko, Baltimore, MD (US)

(21) Appl. No.: 19/135,020

(22) PCT Filed: Dec. 4, 2023
(86) PCT No.: PCT/US2023/082368

§ 371 Date: Jun. 2, 2025

Related U.S. Application Data
(60) Provisional application No. 63/385,885, filed on Dec. 2, 2022.

(51) Int. Cl. A61K 49/00; A61K 31/137; A61K 47/02; A61K 47/12; A61K 47/26; C07C 59/21

(52) U.S. Cl. CPC A61K 49/0002; A61K 31/137; A61K 47/02; A61K 47/12; A61K 47/26; C07C 59/21; A61K 2123/00; C07B 2200/05

(57) ABSTRACT

A diagnostic composition is described having a 3-Bromopyruvate (3-BP) molecule having at least one heavy isotope according to formula I wherein R1, R2, and R3 are independently C or ¹³C, R4 and R5 are independently O, ¹⁷O, or ¹⁸O, and R6 and R7 are independently H or deuterium, with the proviso that at least one R2 or R3 is ¹³C, at least one of R4 or R5 is ¹⁷O or ¹⁸O, or at least one of R6 or R7 is deuterium. The composition further includes at least one sugar to stabilize the 3-BP molecule by substantially preventing the 3-BP molecule from hydrolyzing and a biological buffer present in an amount sufficient to at least partially deacidify the 3-BP molecule and to neutralize metabolic by-products of the 3-BP molecule.

### PRIORITY DATA

This application claims the benefit of U.S. Provisional Patent Application Ser. No. 63/385,885, filed on Dec. 2, 2022, which is incorporated herein by reference.

### BACKGROUND

Cancer is a complex and often life-threatening disease characterized by the uncontrolled growth of abnormal cells in the body. It can affect virtually any organ or tissue and may lead to a range of physical and psychological effects on patients. General cancer treatments aim to eradicate or control cancer cells, with the primary options being surgery, radiation therapy, chemotherapy, targeted therapy, immunotherapy, and hormone therapy.

In addition to the immediate effects of cancer and associate cancer therapeutics, survivors may also experience long-term or late-onset effects, such as organ damage, fertility issues, and secondary cancers. The management of these effects is an essential part of cancer care and often requires a multidisciplinary approach involving healthcare providers from various specialties. Understanding and addressing the side effects of cancer treatment are crucial to improving the overall quality of life for cancer patients and survivors.

### BRIEF DESCRIPTION OF THE DRAWINGS

FIG. 1A illustrates data from a control molecule of 3-BP in accordance with an example embodiment;

FIG. 1B illustrates further data from a control molecule of 3-BP in accordance with an example embodiment;

FIG. 1C illustrates imaging data from a control molecule of 3-BP in accordance with an example embodiment;

FIG. 2A illustrates data from a molecule of heavy 3-BP in accordance with an example embodiment;

FIG. 2B illustrates further data from a control molecule of heavy 3-BP in accordance with an example embodiment;

FIG. 2C illustrates imaging data from a control molecule of heavy 3-BP in accordance with an example embodiment;

FIG. 3A illustrates data from a molecule of heavy 3-BP in accordance with an example embodiment;

FIG. 3B illustrates further data from a control molecule of heavy 3-BP in accordance with an example embodiment;

FIG. 3C illustrates imaging data from a control molecule of heavy 3-BP in accordance with an example embodiment;

FIG. 4A illustrates data from a molecule of heavy 3-BP in accordance with an example embodiment;

FIG. 4B illustrates further data from a control molecule of heavy 3-BP in accordance with an example embodiment;

FIG. 4C illustrates imaging data from a control molecule of heavy 3-BP in accordance with an example embodiment;

FIG. 5A illustrates data from a molecule of heavy 3-BP in accordance with an example embodiment;

FIG. 5B illustrates further data from a control molecule of heavy 3-BP in accordance with an example embodiment;

FIG. 5C illustrates imaging data from a control molecule of heavy 3-BP in accordance with an example embodiment;

FIG. 6A illustrates data from a molecule of heavy 3-BP in accordance with an example embodiment;

FIG. 6B illustrates further data from a control molecule of heavy 3-BP in accordance with an example embodiment;

FIG. 6C illustrates imaging data from a control molecule of heavy 3-BP in accordance with an example embodiment;

FIG. 7A illustrates data from a molecule of heavy 3-BP in accordance with an example embodiment;

FIG. 7B illustrates further data from a control molecule of heavy 3-BP in accordance with an example embodiment;

FIG. 7C illustrates imaging data from a control molecule of heavy 3-BP in accordance with an example embodiment;

FIG. 8A illustrates data from a molecule of heavy 3-BP in accordance with an example embodiment;

FIG. 8B illustrates further data from a control molecule of heavy 3-BP in accordance with an example embodiment;

FIG. 8C illustrates imaging data from a control molecule of heavy 3-BP in accordance with an example embodiment;

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, compounds, ingredients, etc. In other instances, well-known materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including.” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.010%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second.” “third,” “fourth.” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art. For example, a data region that has an “increased” risk of corruption can refer to a region of a memory device which is more likely to have write errors to it than other regions in the same memory device. A number of factors can cause such increased risk, including location, fabrication process, number of program pulses applied to the region, etc.

The compositions of the present invention may include a pharmaceutically acceptable carrier and other ingredients as dictated by the particular needs of the specific dosage formulation. Such ingredients are well known to those skilled in the art. See for example, Gennaro, A. Remington: The Science and Practice of Pharmacy 19^{th} ed. (1995), which is incorporated by reference in its entirety.

As used herein. “administration,” and “administering” refer to the manner in which a composition is presented to a subject. Administration can be accomplished by various art-known routes such as enteral, parenteral, transdermal, and the like, including combinations thereof in some cases. Thus, an enteral administration can be achieved by drinking, swallowing, chewing, sucking of an oral dosage form comprising an active agent or other compound to be delivered. Parenteral administration can be achieved by injecting a drug composition intravenously, intra-arterially, intramuscularly, intrathecally, subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

As used herein, “subject” refers to a mammal that may benefit from the administration of a drug composition or method of this invention. Examples of subjects include humans, and other animals such as horses, pigs, cattle, sheep, goats, dogs (felines), cats (canines), rabbits, rodents, primates, and aquatic mammals. In one embodiment, subject can refer to a human.

As used herein, “effective amount” or “therapeutically effective amount,” or similar terms, refers to a non-toxic but sufficient amount of a drug to achieve therapeutic results in treating a condition for which the drug is known to be effective or has been found to be effective as disclosed herein. Various biological factors may affect the ability of a delivered substance to perform its intended task or the amount of drug needed to provide a therapeutic result. Therefore, an “effective amount” or “therapeutically effective amount” may be dependent on such biological factors. The determination of an effective amount or therapeutically effective amount is well-within the ordinary skill in the art of pharmaceutical and medical sciences based on known techniques in the art as well as the present disclosure. See for example, Curtis L. Meinert & Susan Tonascia, Clinical Trials: Design. Conduct, and Analysis, Monographs in Epidemiology and Biostatistics, vol. 8 (1986).

As used herein. “drug,” “active agent,” “bioactive agent,” “pharmaceutically active agent,” “therapeutically active agent” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, or compounds significantly related thereto, including without limitation, prodrugs, active metabolites, isomers, and the like. The terms “cellular energy inhibitor,” “glycolysis inhibitor,” “mitochondrial inhibitor,” and the like, are considered to be active agents.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

As used herein, “cellular energy inhibitor” refers to a compound that inhibits ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a compound that inhibits, reduces, or stops, glycolysis in a cell.

As used herein. “mitochondria inhibitor” refers to a compound that inhibits, reduces, or stops mitochondrial production of ATP in a cell.

As used herein, the terms “dosage form,” “formulation,” and “composition” are used interchangeably and refer to a mixture of two or more compounds, elements, or molecules. In some examples, the terms “dosage form,” “formulation.” and “composition” may be used to refer to a mixture of one or more active agents with a carrier and/or other excipient.

As used herein, “carrier” or “pharmaceutically acceptable carrier” refers to a substance with which a drug may be combined to achieve a specific dosage formulation for delivery to a subject. In some examples, a carrier may or may not enhance drug delivery. As a general principle, carriers do not react with the drug in a manner that substantially degrades or otherwise adversely affects the drug, except that some carriers may react with a drug to prevent it from exerting a therapeutic effect until the drug is released from the carrier. Further, the carrier, or at least a portion thereof must be physiologically suitable for administration into a subject along with the drug.

As used herein, “admixed” means that at least two components of the composition can be partially or fully mixed, dispersed, suspended, dissolved, or emulsified in one another. In some cases, at least a portion of the drug may be admixed in at least one carrier substance.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

3-Bromopyruvate (3-BP) is an anticancer agent that kills at least a majority of cancer cell types by targeting key energy metabolism (energy production) centers of cancer cells. This metabolic approach of using 3-BP combat cancer has proven effective in treating cancer and improving patient prognosis.

Adenosine triphosphate (ATP) is an organic molecule that transfers energy to cells to maintain their viability and to drive cellular processes. Specifically, ATP is a coenzyme that works with various enzymes to transfer energy to cells by releasing its phosphate groups. There are generally two ATP production pathways inside of cells, glycolysis and oxidative phosphorylation. Oxidative phosphorylation is a metabolic pathway present in nearly all aerobic organisms, whereby cells utilize various enzymes to oxidize nutrients to release chemical energy and produce ATP. In eukaryotes, such as humans, oxidative phosphorylation also takes place inside mitochondria, energy production factories within cells.

Glycolysis, which generally occurs in the cytoplasm of cells, is a metabolic pathway that converts glucose into pyruvate, generating ATP from the energy released in the process. In noncancerous (i.e., normal) cells under normal conditions, about 5% of the total ATP production is derived from glycolysis and about 95% from oxidative phosphorylation. In cancer cells, the energy production by glycolysis can be significantly increased (up to 60%). This dramatic increase in glycolysis in cancer cells results in a significant increase in lactic acid production.

Most cancers (>90%) exhibit this common metabolic phenotype. This is called the “Warburg Effect”, i.e., significant increase in glycolysis in cancer cells even in the presence of oxygen. On detection method frequently used clinically to identify cancer is Positron Emission Tomography (PET), which detects cancers based on the Warburg effect metabolic phenotype, i.e., the “Warburg effect”. Cancer cells that exhibit the “Warburg effect” pump out lactic acid generated by the process via monocarboxylate transporter. The number of these transporters (considered as doors or gates) in cancer cells is much greater than in normal cells.

The present technology relates to, among other things, the treatment of cancer with a 3-bromopyruvate molecule that has been modified with one or more heavy isotopes. Such “heavy 3-BP” molecules effectively treat most if not all cancers and allow simultaneous diagnostic imaging of the anticancer therapeutic (heavy 3-BP). Heavy 3-BP (a lactic acid analog) has a chemical structure that is similar to the lactic acid chemical structure and thus can readily enter cancer cells. In contrast to cancer cells, normal cells express few lactic acid transporters. Heavy 3-BP (and 3-BP in general) has little to no effect on normal cells (i.e., noncancerous cells) due to the fact that normal cells express very few lactic acid transporters compared to cancerous cells. Heavy 3-BP is thus taken up primarily by cancer cells, where it disrupts the oxidative phosphorylation and glycolysis pathways, leading to a rapid decrease in energy production and subsequent death of the affected cells.

In general, various techniques are currently used to determine the efficacy of potential cancer treatments, including, biopsies, blood tests, and general functional assessments, and direct imaging, to name a few. Biopsies often entail analysis that involves examining cancer tissue samples microscopically to observe changes in cancer cells post-treatment, while blood tests monitor for tumor markers in the blood which can indicate the continued presence or progression of certain cancer types. Function measurements are a generally observational testing to determine how well a patient functions and manages daily activities, which can provide indirect evidence of treatment effectiveness. Direct imaging of cancer utilizes numerous imaging techniques to monitor tumor size and metastasis in response to a cancer treatment, including MRI. CT scans, PET scans, and X-rays. While direct imaging has the capacity to examine cancerous tumors in vivo, such is limited to macroscopic tumor size and its reduction over time. In some cases, contrast has been used in conjunction with various cancer therapeutics; however, the fact that a cancer therapeutic and a contrast agent are very different materials and require separate administrations, the localized correlation between the two may be vastly different, particularly give the different uptake mechanisms inherent between cancer and normal cells. The aforementioned techniques for monitoring cancer treatment effectiveness are, therefore, not suitable for real-time treatment analysis, and primarily only provide potential effectives of a cancer therapeutic from days to months following administration. In other words, the efficacy of a traditional cancer treatment is generally not known until an anticancer agent has been given a sufficient time to cause a measurable effect in the cancer, such as the measurable effects described above.

When treating a cancer with an anticancer agent, the degree of uptake and the resulting distribution of the anticancer agent within the cancer and particularly in the surrounding tissue is not directly immediately ascertainable, particularly in real time. In addition to timing issues, such traditional chemotherapeutic efficacy techniques (including radiological and cytological testing, biomarker assays, and the overall physiological response of the patient, etc.) each have potential flaws—radiological testing, for example, presumes that an appropriate radiological marker has been selected for imaging the cancerous tissue. While a radiological signal may be associated with a cancerous region, the presumption that the radiological marker is binding to all of the various cancer cell types present in that region is not a surety. For example, assuming a tumor is made up of 2 cancer cell types, cell type A and cell type B. As cell type A is the predominant cell type, a prior biopsy revealed only the presence of that cell type. Treatment with an anticancer agent effective against cell type A will likely reduce the tumor, as evidence by the distribution of a radiological marker specific for cell type A. Further cytological tests and biomarker assays, directed again to the predominant cell type A, are all in agreement with the radiological results. The patient is showing signs of improvement, as the predominant cell type A had been greatly reduced or eliminated. However, cell type B, which was not detected by the original biopsy or the subsequent testing following the use of the anticancer agent, is now free to proliferate. The resurgence of the cancer may lead clinicians to the mistaken belief that the cancer has now become resistant to the given anticancer agent.

The present disclosure describes a novel molecule and associated cancer therapeutic techniques for allowing the immediate visualization of the cancer therapeutic distribution across affected areas, both in cancerous and noncancerous tissue. This visualization allows a clinician to visualize in real time the borders of tumor tissue and the presence, and to verify the presence or lack thereof of the cancer therapeutic in noncancerous surrounding tissue. Such a visualization of tumor borders in real time allows tumor size to be more accurately determined, which allows the efficacy of the cancer therapeutic to be directly evaluated. This can also be crucial to facilitate a more rapid determination of cancer therapeutic efficacy without the need to wait for a sufficient time to elapse to allow traditional imaging techniques to evaluate tumor size.

The present disclosure provides a molecule that can be readily imaged in tissue in vivo once administered. I addition to acting as a contrast agent itself, the molecule additionally had cancer therapeutic properties itself. In this manner, the administration of the facilitates the destruction of cancer cells, while at the same time allowing the real time imaging of the localization of the molecule in the tumor and in any surrounding tissue, if applicable.

In one example, the cancer therapeutic can be a halo-bromopyruvate molecule such as 3-promopyruvate, including salts and acids thereof (collectively referred to herein as 3-BP), which have at least one heavy isotope replacement atom (collectively referred to herein as heavy 3-BP) that allows for diagnostic imaging, diagnostic imaging and simultaneous cancer treatment, or cancer treatment. Additionally, heavy 3-BP molecules are more stable, both physiologically and in storage. Below are nonlimiting examples of heavy 3-BP molecules. It is noted that the below are merely a selection of heavy 3-BP examples, and any such combination of atoms and/or heavy isotopes of those atoms are within the present scope.

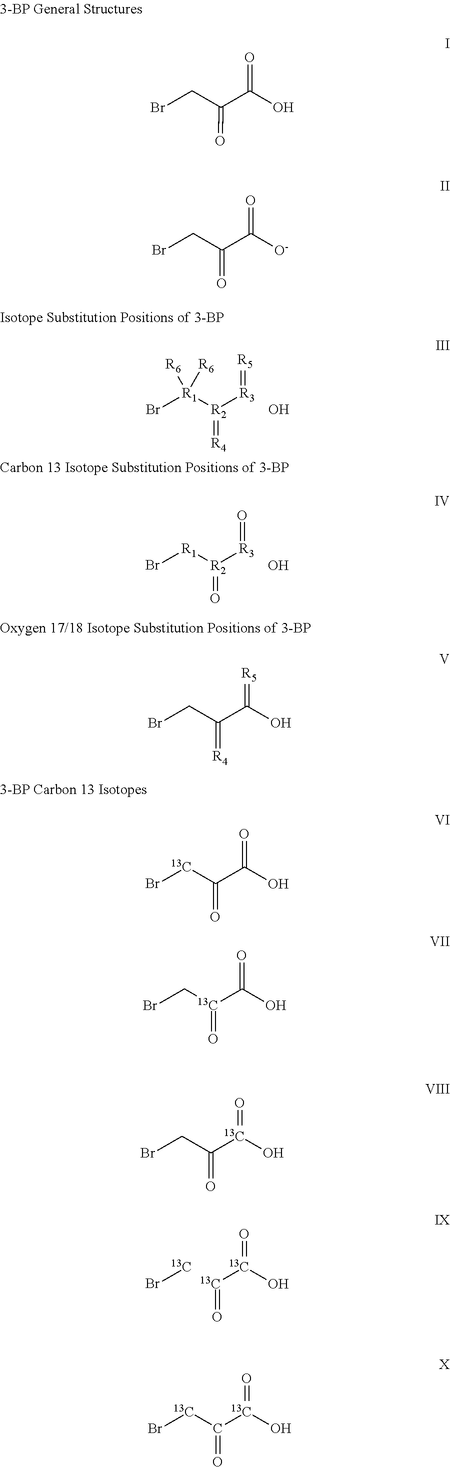

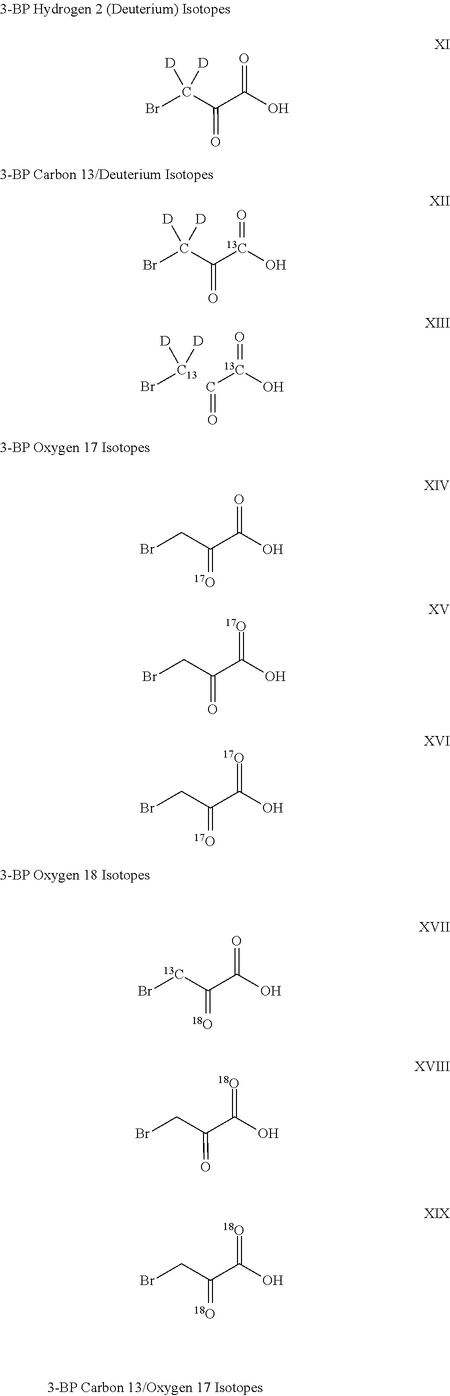

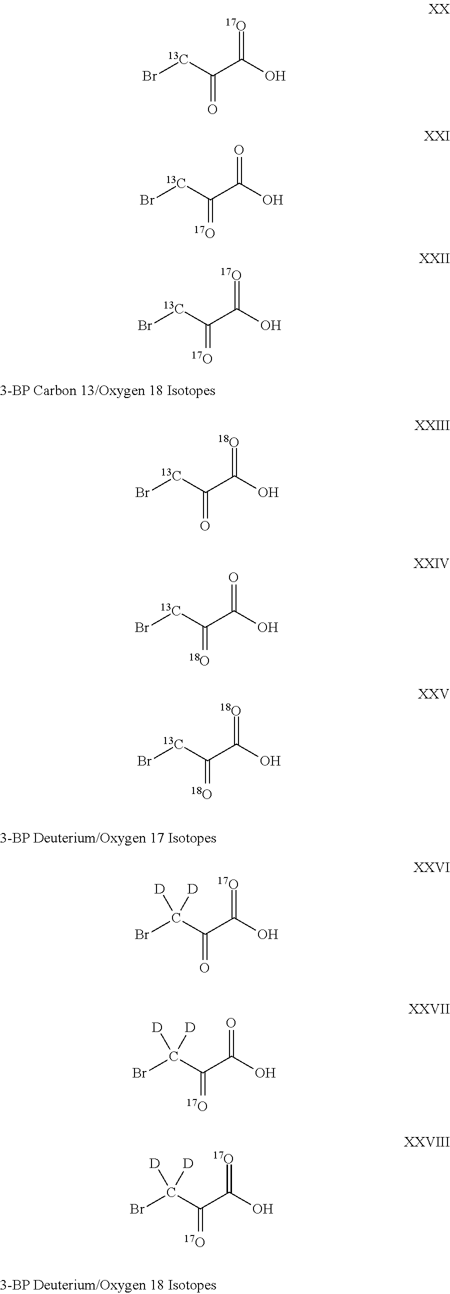

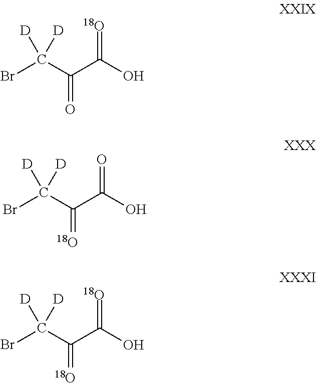 [SMILES: [2H]C([2H])(Br)C(=O)C(O)=[18O] ; [2H]C([2H])(Br)C(=[18O])C(=O)O ; [2H]C([2H])(Br)C(=[18O])C(O)=[18O]]

As a diagnostic molecule, heavy 3-BP can be delivered to a subject and imaged in real time, thus providing an immediate view of the distribution of 3-BP in relation to cancerous tissue and surrounding normal tissue. Additionally, because 3-BP generally does not enter normal cells but has the capacity to enter most if not all cancers, imaging of heavy 3-BP can allow the identification of cancer cell types that may have been missed in a biopsy or other diagnostic procedure. Furthermore, heavy 3-BP allows a precise localization of a cancer boundary, which can be useful in determining the progression or regression of the cancerous tissue. Such boundary localization additionally allows doctors to have a more accurate view of, for example, regions to be excised by surgery or regions to be irradiated by radiation therapy.

Heavy 3-BP can be imaged by any technique known to detect heavy isotopes. Nonlimiting examples include magnetic resonance imaging (MRI), magnetic resonance spectroscopy (MRS), single-photon emission computed tomography (SPECT), computed tomography (CT), positron emission tomography (PET), and the like, including combinations thereof.

In another example, heavy 3-BP can be used as a simultaneous diagnostic and cancer therapy. In a cancer therapeutic procedure, heavy 3-BP can be delivered to a subject in order to eradicate the cancer. The progress of the therapy and the distribution of the heavy 3-BP within the cancer provides therapeutic information that can be used to, for example, modify the treatment procedure much more quickly, and in some cases more effectively, compared to therapies that require a separate diagnostic performed after a given cancer therapy has been administered.

Different molecules having different configurations of isotopes in heavy 3-BP can also be used to treat cancers at a different rate that is dependent on the isotope configuration. For example, isotope positioning can affect how tightly the pyruvate molecule binds to the bromide molecule. As such, the timing of the release of bromide can be influenced by the isotope configuration in the heavy 3-BP molecule.

In one example, a diagnostic cancer treatment composition includes a 3-bromopyruvate (3-BP) molecule having at least one heavy isotope according to formula I

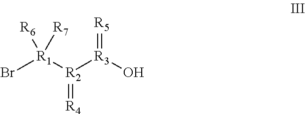

wherein R1, R2, and R3 are independently C or ¹³C, R4 and R5 are independently O, ¹⁷O, or ¹⁸O and R6 and R7 are independently H or deuterium, with the proviso that at least one R2 or R3 is C, at least one of R4 or R5 is ¹⁷O or ¹⁸O, or at least one of R6 or R7 is deuterium. Additionally, the diagnostic cancer treatment composition can comprise at least one sugar to stabilize the 3-BP by substantially preventing the inhibitor from hydrolyzing. The diagnostic cancer treatment composition can further comprise a hexokinase inhibitor. Further, the diagnostic cancer treatment composition can also comprise a biological buffer that is present in an amount sufficient to at least partially deacidify the 3-BP and neutralize metabolic by-products of the 3-BP. The diagnostic cancer treatment composition can further comprise a glycolysis inhibitor.

The inventor has recognized the need to provide safe and efficacious compositions that allow for treatment of cancers. The 3-BP can be stabilized by the use of at least one sugar such that the sugar substantially prevents hydrolysis of the 3-BP. In this way, the sugar can stabilize the 3-BP for at least 1 hour such that at least 50% of the inhibitor does not hydrolyze. In another embodiment, the at least one sugar can stabilize the 3-BP for at least 1 hour and prevent at least 95% of the inhibitor from hydrolyzing. In yet another embodiment, the at least one sugar can stabilize the 3-BP for at least 2 hours such that at least 95% of the inhibitor does not hydrolyze.

The diagnostic cancer treatment compositions disclosed herein generally include a compound as described by formula (I). The diagnostic cancer treatment composition can comprise the 3-HP in a concentration from about 0.1 mM to about 25.0 mM. In one embodiment, the diagnostic cancer treatment composition can comprise the 3-BP in a concentration from about 1.0 mM to about 10.0 mM.

While the diagnostic cancer treatment composition generally comprises at least one sugar, in one embodiment, the diagnostic cancer treatment composition can comprise other sugars, such as a second sugar. In another embodiment, the diagnostic cancer treatment composition can comprise a third sugar. The sugars described herein can include their analogues. In one embodiment, the sugar can be gluconic acid. In another embodiment, the sugar can be glucuronic acid. At least one of the sugars can be a five-carbon sugar. In one embodiment, at least two of the sugars can be five-carbon sugars. The five-carbon sugars can be independently selected from the group consisting of mannitol, erythritol, isomalt, lactitol, maltitol, sorbitol, xylitol, dulcitol, ribitol, inositol, sorbitol, and combinations thereof. In one embodiment, at least one of the sugars can be glycerol. In another embodiment, the sugars can be glycerol, inositol, and sorbitol. The diagnostic cancer treatment composition can comprise glycerol in a range from about 0.1 wt % to about 3 wt %, inositol in a range from about 1 wt % to about 5 wt %, and sorbitol in a range from about 30 wt % to about 50 wt %. Additionally, each of the sugars may be added in a volume up to a maximum solubility of the sugar in the formulation or composition.

The sugars described herein can be any isomeric form. In one embodiment, the diagnostic cancer treatment compositions described herein can include the less biologically active form of the sugar as compared to its isomer. In one aspect, the less biologically active sugar can be the L-enantiomer sugar. However, if the D-enantiomer sugar is found to be less biologically active as compared to its L form, then the D form can be used. In one embodiment, such sugars can function as a glycolytic inhibitor.

In one embodiment, the diagnostic cancer treatment composition can comprise the at least one sugar in a concentration from about 0.1 mM to about 250 mM. In another embodiment, the diagnostic cancer treatment composition can comprise the at least one sugar in a concentration from about 0.5 mM to about 25 mM.

Generally, the diagnostic cancer treatment composition can comprise a glycolysis inhibitor. In one embodiment, the glycolysis inhibitor can be 2-deoxglucose. The diagnostic cancer treatment composition can comprise the glycolysis inhibitor in a concentration from about 0.1 mM to about 25.0 mM. In one embodiment, the diagnostic cancer treatment composition can comprise the glycolysis inhibitor in a concentration from about 1 mM to about 5 mM.

Generally, the diagnostic cancer treatment composition can include a biological buffer that is present in an amount sufficient to at least partially deacidify the 3-BP and neutralize metabolic by-products of the 3-BP. In one embodiment, the biological buffer can be selected from the group consisting of a citrate buffer, a phosphate buffer, and an acetate buffer. In another embodiment, the biological buffer can be a citrate buffer. In still another embodiment, the biological buffer can be sodium citrate.

As discussed herein, the 3-BP is delivered to a cancer cell and is taken up by the cell. After metabolism of the 3-BP, the 3-BP can cause by-products. In one embodiment, the by-product can be a hydrogen halide. Additionally, the hydrogen halide can be hydrogen bromide or hydrogen iodide. In one embodiment, the hydrogen halide can be hydrogen bromide.

The diagnostic cancer treatment composition can comprise the biological buffer in a concentration of from about 0.1 mM to about 200 mM. In one embodiment, the diagnostic cancer treatment composition can comprise the biological buffer in a concentration of from about 1 mM to about 20 mM. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6.

In addition to the above components, the diagnostic cancer treatment compositions described herein can further comprise a halo monocarboxylate compound. In the cases where the halo monocarboxylate compound can function to inhibit glycolysis and mitochondria function, the halo monocarboxylate can be considered a cellular energy inhibitor. In one embodiment, the halo monocarboxylate compound can be a halo two-carbon monocarboxylate compound. The halo two-carbon monocarboxylate compound can be selected from the group consisting of 2-fluoroacetate, 2-chloroacetate, 2-bromoacetate, 2-iodoacetate, and mixtures thereof. In one embodiment, the halo two-carbon monocarboxylate compound can be 2-bromoacetate. The diagnostic cancer treatment composition can comprise the halo two-carbon monocarboxylate compound in a concentration from about 0.01 mM to about 5.0 mM. In one embodiment, the diagnostic cancer treatment composition can comprise the halo two-carbon monocarboxylate compound in a concentration from about 0.1 mM to about 0.5 mM.

Additionally, the halo monocarboxylate compound can be a halo three-carbon monocarboxylate compound. In one embodiment, the halo three-carbon monocarboxylate compound can be selected from the group consisting of 3-fluorolactate, 3-chlorolactate, 3-bromolactate, 3-iodolactate, and mixtures thereof. The diagnostic cancer treatment composition can comprise the halo three-carbon monocarboxylate compound in a concentration from about 0.5 mM to about 250 mM. In one embodiment, the diagnostic cancer treatment composition can comprise the halo three-carbon monocarboxylate compound in a concentration from about 10 mM to about 50 mM.

The diagnostic cancer treatment compositions described herein can further comprise an antifungal agent and/or antibacterial agent. In one embodiment, the diagnostic cancer treatment composition can individually comprise the antifungal agent and/or antibacterial agent in a concentration from about 0.01 mM to about 5.0 mM. In another embodiment, the diagnostic cancer treatment composition can individually comprise the antifungal agent and/or antibacterial agent in a concentration from about 0.05 mM to about 0.5 mM.

The diagnostic cancer treatment compositions described herein can further comprise a mitochondrial inhibitor. The mitochondrial inhibitor can be selected from the group consisting of: oligomycin, efrapeptin, aurovertin, and mixtures thereof. In one embodiment, the diagnostic cancer treatment composition can comprise the mitochondrial inhibitor in a concentration from about 0.001 mM to about 5.0 mM. In another embodiment, the diagnostic cancer treatment composition can comprise the mitochondrial inhibitor in a concentration from about 0.01 mM to about 0.5 mM.

In addition to the above concentrations, the diagnostic cancer treatment compositions can have various ratios of the components described herein. In one embodiment, the 3-BP and biological buffer can be present in a ratio ranging from 1:1 to 1:5 by mM. In another embodiment, the 3-BP and glycolysis inhibitor can be present in a ratio ranging from 5:1 to 1:1 by mM. In still another embodiment, the 3-BP and the at least one sugar are present in a ratio ranging from 1:1 to 1:5 by mM. In yet another embodiment, the 3-BP and the halo two-carbon monocarboxylate compound can be present in a ratio ranging from 20:1 to 4:1 by mM. In still yet another embodiment, the 3-BP to mitochondrial inhibitor can be present in a ratio ranging from 20:1 to 40:1 by mM.

As described above, the present diagnostic cancer treatment compositions can comprise antifungal agents, antibiotics, glycolysis inhibitors, inhibitors of mitochondria, sugars, and biological buffers. Examples of such agents include, but are not limited to, amphotericin B, Efrapeptin, doxorubicin, 2-deoxyglucose (2DOG), d-lactic acid, analogs of 2DOG, dicholoracetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. The active agents of the diagnostic cancer treatment composition can include the 3-BP, the glycolysis inhibitor, the mitochondria inhibitor, the halo monocarboxylate compound, the antifungal agent, and the antibiotic agent.

The diagnostic cancer treatment composition can further include various additives. In one embodiment, the diagnostic cancer treatment compositions can include immune system modulators and/or immune system boosters. Such immune system modulators and/or immune system boosters can include d-lactic acid, epinephrine, brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, antibodies against nagalase, etc. Without being bound by any particular theory, flavonoids may have indirect anti-cancer effects. Specifically, increase in antioxidant capacity of blood seen after the consumption of flavonoid-rich foods is not caused directly by flavonoids themselves, but most likely is due to increased uric acid levels that result from metabolism of flavonoids. The body sees them as foreign compounds and is trying to get rid of them. This process of removing unwanted compounds includes Phase II enzymes that also help eliminate mutagens and carcinogens, and therefore may be of value in cancer prevention. Therefore, flavonoids could also induce mechanisms that help kill cancer cells and inhibit tumor invasion. In one embodiment, the present compositions can include d-lactic acid. In another embodiment, the present compositions can include epinephrine.

In one embodiment, the additives to the diagnostic cancer treatment compositions can include phospholipids including liposomes and nanoparticles. The liposomes or nano-particles can incorporate annexin-A5 molecules or antibodies against phosphatidylserine. In this way, the rate of 3BP release can be controlled and its delivery can be targeted to cancer cells upon its administration. Liposomes can have a natural ability to target cancer. Without intending to be bound by any particular theory, the endothelial wall of all healthy human blood vessels is encapsulated by endothelial cells that are bound together by tight junctions. These tight junctions can stop any large particle(s) in the blood from leaking out of the vessel. Generally, tumor vessels do not contain the same level of seal between cells and are diagnostically leaky. In one embodiment, liposomes of certain sizes, typically less than 400 nm, can rapidly enter tumor sites from the blood, but are kept in the bloodstream by the endothelial wall in healthy tissue vasculature. Additionally, the additives to the diagnostic cancer treatment compositions can include L-Lactate dehydrogenase or D-Lactate Dehydrogenase (or both forms of the enzymes) as well as nicotinamide adenine dinucleotides (NAD⁺), which can be included in the present formulations to decrease the blood lactate concentration as well as the lactate concentration near tumor sites. By decreasing the blood lactate concentration in cancer patients, the highly glycolytic innate immune system can work appropriately.

In one embodiment, the additives to the diagnostic cancer treatment compositions can include less biologically active amino acids as compared to their isomers to facilitate cancer cell starvation. In one aspect, the less biologically active amino acid can be a D-amino acid. However, if the L-amino acid is less biologically active than the D-form, the L-amino acid can be used.

In one embodiment, the additives to the diagnostic cancer treatment compositions can include

inhibitors for DNA replication; inhibitors for DNA binding; and inhibitors for DNA transcription. In another embodiment, the additives to the diagnostic cancer treatment compositions can include inhibitors for cell cycle, growth and/or proliferation. In yet another embodiment, the additives to the diagnostic cancer treatment compositions can include inhibitors for signal transduction pathways. In yet another embodiment, the additives to the diagnostic cancer treatment compositions can include inhibitors for angiogenesis. In yet another embodiment, the additives to the diagnostic cancer treatment compositions can include small RNAs that interfere with normal gene control including antisense RNA, micro RNA, small hairpin RNA, short hairpin RNA, small interfering RNA. In yet another embodiment, the additives to the diagnostic cancer treatment compositions can include vitamin C; nutritional supplements including vitamins, CoQ10, flavonoids, free fatty acid, alpha lipoic acid, acai, goji, mango, pomegranate, L-carnitine, selenium; etc.

In addition to the active agent(s), the composition can also include a pharmaceutically acceptable carrier. The carrier can be a single composition, or a mixture of compositions. Additionally, the carrier can take the form of an encapsulation coat, an absorbing agent, a coating substance, a controlled release device, a release modifying agent, surfactants, or a combination thereof. In some aspects, the carrier can comprise about 1 wt % to about 99 wt % of the total composition. In one embodiment, the carrier can comprise about 5 wt % to about 95 wt % of the total formulation. In another embodiment, the carrier can comprise about 20 wt % to about 80 wt %. In yet a further embodiment, the carrier can comprise about 30 wt % to about 60 wt %. In one embodiment, the carrier can be admixed with the active agent(s). In another embodiment, the carrier can adsorb, entrap, or encapsulate at least a portion of the active agent(s).

Non-limiting examples of compounds that can be used as at least a part of the carrier include without limitation: cetyl alcohol and its esters; stearic acid and its glycerol esters, polyoxyethylene alkyl ethers; polyethylene glycol; polyglycolyzed glycerides; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers: polyglycerol fatty acid esters: proteins: polyoxyethylene glycerides: polyoxyethylene sterols, derivatives, and analogues thereof; polyoxyethylene hydrogenated vegetable oils; reaction mixtures of polyols with at least one member of the group consisting of fatty acids, glycerides, vegetable oils, hydrogenated vegetable oils, and sterols: tocopherol derivatives, sugar esters; sugar ethers; sucroglycerides; waxes, shellac, pharmaceutically acceptable salts thereof, and mixtures thereof.

Non-limiting examples of release modifying agents include without limitation: polyethylene glycols having a weight average molecular weight of about 1000 and more, carbomer, methyl methacrylate copolymers, methacrylate copolymers, hydroxypropyl methyl cellulose, hydroxypropyl cellulose, cellulose acetate phthalate, ethyl cellulose, methyl cellulose and their derivatives; ion-exchange resin; mono-, di-, tri-esters of fatty acids with glycerol; tocopherol and its esters; sucrose esters with fatty acids; polyvinyl pyrollidone: xanthan gums: cetyl alcohol; waxes; fats and oils, proteins, alginate, polyvinyl polymers, gelatins, organic acids, and their derivatives and combinations thereof.

In one embodiment, the carrier can include at least one of celluloses; carbomers; methacrylates; dextrins; gums: inorganic carbonates or salts of calcium or magnesium or both; fatty acid esters; gelatin; lactoses; maltoses; mono-, di- or triglycerides; oils; polyethylene glycols; polyethylene oxide co-polymers: proteins; resins; shellac; silicates; starches; sugar stearates; partially or fully hydrogenated vegetable oils; waxes; and combinations thereof.

In yet another embodiment, the carrier can include at least one of celluloses; carbomers; methacrylates; inorganic carbonates or salts of calcium; inorganic carbonates or salts of magnesium; fatty acids; fatty acid esters; gelatin; lactoses; polyethylene glycol; polyethylene oxide co-polymers: silicates; partially or fully hydrogenated vegetable oils, and combinations thereof.

In yet a further embodiment, the carrier can include at least one of microcrystalline cellulose; hydroxypropyl methylcellulose; ethyl cellulose; silicon dioxide; magnesium aluminosilicate; lactose; xanthan gum; stearic acid; glyceryl distearate; hydrogenated vegetable oil; and combinations thereof.

The formulation, including any dosage form, can include other components or additives. Such additional components and additives are optional. In one aspect, the additive can be a solid at room temperature and have a melting point or range that is greater than about 40° C. Non-limiting examples of additives that can be included in the systems of the present invention include without limitation: fillers such as lactoses, starches, sugars, celluloses, calcium salts, silicon oxides, metallosilicates and the like; disintegrants such as starch glycolate, lauryl sulfate, pregelatinized starch, croscarmellose, crospovidone and the like; binders such as pyrrolidones, methacrylates, vinyl acetates, gums, acacia: tragacanth; kaolins; carrageenan alginates, gelatins and the like; cosolvents such as alcohols, polyethylene glycols having average molecular weight of less than 1000, propylene glycols and the like; surface tension modifiers such as hydrophilic or amphiphlic surfactants; taste-masking agents; sweeteners: microencapsulating agents; process aids such as lubricants, glidants, talc, stearates, lecithin and the like; polymeric coating agents; plasticizers; buffers; organic acids: antioxidants: flavors; colors; alkalizers; humectants; sorbitols; mannitols; osmotic salts; proteins; resins; moisture repelling agents; hygroscopic agents; desiccants; and combinations thereof.

The formulations of the present invention can be formulated into a variety of oral dosage forms including, but not limited to two piece hard gelatin capsules, soft gelatin capsules, beads, beadlets, granules, spherules, pellets, microcapsules, microspheres, nanospheres, nanocapsules, tablets, or combinations thereof. Other forms known to those of ordinary skill in the art may also be used. In one aspect, the oral dosage form may be a capsule or tablet. In another embodiment the oral dosage form may include a multi-component dosage form such as beads in a capsule, a capsule or capsules within a capsule, a tablet or tablets in a capsule, or a multilayer tablet. It is noteworthy that, when the formulation includes multiple dosage forms, such dosage forms need not be the same. Further, such dosage forms may not be physically present together.

The dosage form. e.g. tablet, may be coated or enrobed with a hydrophilic or a hydrophobic coat material known in the art. In one embodiment, the coat can be a film coat, sugar coat, enteric coat, semipermeable coat, sustained release coat, delayed release coat, osmotic coat and the like. In a further embodiment, the coating material can be a cellulose, gelatin, methacrylate, polyvinyl acetate, povidone, polyethylene glycol, polyethylene oxide, poloxamers, carbomers, shellac, phthalate and the like and their derivatives and combinations thereof. In another embodiment, the coat is a dry powder coat. In one embodiment, the tablet can be a matrix tablet. It is noteworthy that, when present, the coat can be considered as part, or all, of the carrier component of the formulation.

In addition to the compositions described herein, a method for the treatment of cancer can comprise administering to a subject the diagnostic cancer treatment compositions as described herein in a therapeutically effective amount. The diagnostic cancer treatment composition can be administered to the subject when the subject's blood insulin/glucagon ratio is in the range of about 1 to about 10. Additionally, the diagnostic cancer treatment composition can be administered to the subject after fasting for at least 4 hours. In one embodiment, the diagnostic cancer treatment composition can be administered to the subject after fasting for 6 hours, and in another embodiment, after fasting for 8 hours. Additionally, the diagnostic cancer treatment composition can be administered to the subject after fasting for 2 hours. It is noted that such times are not intended to be limiting, and that in one embodiment, the amount of time fasting can be such that the subject's blood insulin/glucagon ratio is in the range of about 2 to about 5.

In addition, the method of administration can be selected from the group consisting of: inter-arterially, intravenously, inter-peritoneally, inhalation, intra-tumorally, orally, topically, and subcutaneously. In one embodiment, the administration can be inter-arterially. The diagnostic cancer treatment compositions can also be delivered by use of a feeding tube. Intra-tumorally delivery methods can include technologies involving a bronchoscope, an endoscope, and/or a colonoscopy, suppository to any openings, eye drops, nose drops, and ear drops. In one embodiment, the administration can be by intranasal delivery. Intranasal delivery can be used to bypass the blood brain barrier and can be particularly effective for tumors in the brain and/or spinal cord. In another embodiment, the administration can be by suppository. Suppository administration can be used for tumors in proximity to the rectal/anal area. Additionally, if intra-tumorally injection is going to be performed directly to/in the tumor, ultrasound imaging (or other imaging methods) can be used to aid this injection. In one embodiment, the administration can be by direct injection; e.g., to a prostate gland. Additionally, administration can be by an enema containing the composition described herein into the rectum and/or lower intestines. Chronic irrigation to treat obstructive colon, intestinal, or other obstructive cancers, can also be used in conjunction with the compositions described herein. In one embodiment, administration can also be by catheter to treat bladder cancers via the urethra. Further, intravenous delivery can be combined with a hemodialysis apparatus (i.e. kidney dialysis equipment) to destroy the metastatic circulating cancer cells outside of the blood vessels. In addition, both intravenous and inter-peritoneal can be assisted by utilization of a port system. Furthermore, the present diagnostic cancer treatment composition can be immediate release, controlled release, or time controlled release. For time controlled release, the present compositions can delivered by implanting wafers, diamond chips, and other implantable devices near or on the tumor site.

Generally, when the diagnostic cancer treatment composition is administered intra-arterially or intravenously, the administration can be for a duration from about 30 minutes to about 8 hours. In one embodiment, the diagnostic cancer treatment composition can be intra-arterially or intravenously administered for a duration from about 3 hours to about 5 hours. Additionally, the administration of the diagnostic cancer treatment composition can be part of a dosing regimen. In one embodiment, the administration can include a regimen lasting from about 1 week to 24 weeks. In another embodiment, the regimen can last from about 4 weeks to 8 weeks.

Generally, the present diagnostic cancer treatment composition is administered in a therapeutically effective amount as defined herein. In one embodiment, the therapeutically effective amount can include a dosage of, or equivalent to, about 1 mM to about 10 mM of the diagnostic cancer treatment composition in a volume of 25 ml to 1000 ml.

The diagnostic cancer treatment compositions described herein can be used to treat any cancer having increased glycolysis; the metabolic phenotype referred to as the “Warburg Effect”, as described above. In another embodiment, the diagnostic cancer treatment compositions can be used to treat any cancer that can be detected by Positron Emission Tomography (PET), which detects this metabolic phenotype. Examples can include liver, cervical, ovarian, lung, breast, colon, neuroblastoma, medulloblastoma, prostate, skin, pancreatic, childhood fibrolamellar hepatocellular carcinoma (FHCC), hepatocellular carcinoma (HCC), non-small cell lung cancer. The present diagnostic cancer treatment compositions can additionally treat wet cancers.

As such, the present cancers that can be treated with the present diagnostic cancer treatment compositions can be selected from the group consisting of liver, cervical, ovarian, lung, breast, colon, neuroblastoma, medulloblastoma, prostate, skin, pancreatic, childhood fibrolamellar hepatocellular carcinoma (FHCC), hepatocellular carcinoma (HCC), non small cell lung cancer. The present diagnostic cancer treatment compositions have been used to treat human cancer patients having childhood fibrolamellar hepatocellular carcinoma (FHCC), hepatocellular carcinoma (HCC), non small cell lung cancer, colon cancer, breast cancer, and pancreatic cancer. As such, cancers that can be treated with the present diagnostic cancer treatment compositions can be selected from the group consisting of childhood fibrolamellar hepatocellular carcinoma (FHCC), hepatocellular carcinoma (HCC), non small cell lung cancer, colon cancer, breast cancer, pancreatic cancer, and combinations thereof.

In one embodiment, the diagnostic cancer treatment composition can be used to treat liver cancer. In another embodiment, the diagnostic cancer treatment composition can be used to treat cervical cancer. In still another embodiment, the diagnostic cancer treatment composition can be used to treat ovarian cancer. In still another embodiment, the diagnostic cancer treatment composition can be used to treat lung cancer. In still another embodiment, the diagnostic cancer treatment composition can be used to treat breast cancer. In still another embodiment, the diagnostic cancer treatment composition can be used to treat colon cancer. In still another embodiment, the diagnostic cancer treatment composition can be used to treat neuroblastoma. In still another embodiment, the diagnostic cancer treatment composition can be used to treat medulloblastoma. In still another embodiment, the diagnostic cancer treatment composition can be used to treat prostate cancer. In still another embodiment, the diagnostic cancer treatment composition can be used to treat skin cancer. In still another embodiment, the diagnostic cancer treatment composition can be used to treat breast cancer. In still another embodiment, the diagnostic cancer treatment composition can be used to treat pancreatic cancer. In still another embodiment, the diagnostic cancer treatment composition can be used to treat childhood fibrolamellar hepatocellular carcinoma (FHCC). In still another embodiment, the diagnostic cancer treatment composition can be used to treat hepatocellular carcinoma (HCC). In still another embodiment, the diagnostic cancer treatment composition can be used to treat small cell and non small cell lung cancer. In still other embodiments the diagnostic cancer treatment composition can be used to treat vaginal, anal, testicular, nasal, throat, mouth, esophageal, and brain cancers.

### Example: Panc-2 Cells Treated with 3-BP

Pancreatic cancer (PC) is the fourth most lethal cancer in the United States. In 2022, there will be an estimated diagnosis of 62,210 new cases and 49,830 deaths. PC has one of the highest mortality rate amongst all other cancers in both men and women, with a one-year relative survival rate of about 20% and a five-year relative survival rate of 8%. The pancreaticoduodenectomy can increase survival for patients with respectable PC, however, less than 20% of patients are candidates for surgery at time of presentation. Most of the patients are diagnosed with advanced PC, often with regional and distant metastasis. In these advanced cases, chemotherapy and radiation have shown limited tumor control, and PC continues to be refractory to treatment and new treatment options are desperately needed.

3-BP is a promising molecule drug and analog of pyruvic acid that exhibits strong anticancer activity. In pancreatic cancer, the use of 3-nP has demonstrated the therapeutic potential of targeting energy metabolism in cancer cells by inhibiting glycolysis, a major energy producing pathway. Interestingly, 3-Bromo-Pyruvate inhibits expression of Hexokinase II (HK2), resulting in the disruption of glucose metabolism leading to cancer cell death. In current study we have tested anti-tumor potency of 7, 3-BP analogues employing MTS and ATP assay. The percent viability of Pan-2 cells was determined in presence of graded concentration of 3BP analogues 0, 1, 2, 3, 4, 5, 6 & 7.

### Methods:

### MTS Assay:

Panc2 cells were seeded in either 6 well plate (6-6.5×10\hat{ }4 cells/well/2 ml media) or in 12 well plate (3-3.5×10\hat{ }4/1 ml media) or in 96 well plate (2×10′/well/100 ul) in RPMI 1640 with 10% FBS, 0.5% antibiotic media for about 12 hours, in CO2 cell culture incubator at 37\hat{ }0C. The cells were treated initially with KAT 0, 5, 20, 40, 80 and 100 uM for 1, 2, 4, 8 hours and latter in separate experiments with 40 and 60 M for 15, 30, 1 hour. For each treatment KAT was added in a volume maximum 10 ul. For control same amount of KAT buffer was used. At the end of each time point treatment MTS reagent (Cell titer 96 aqueous one solution, Promega, USA) was added to each well according to the manufacturer instruction and incubated for approx. 2 hours. After that 100 ul from each well was transferred to 96 well plate (quartiles for each treatment) and measured absorbance at 490 nm with BIOTEK synergy HT. For 96 well plate reagent was added directly to each well, incubated for 2 hours and measured absorbance at 490 nm.

### APT Assay:

For ATP assay panc2 cells (2×10\hat{ }3/in 100 ul media) were seeded in each well of 96 well luminescence plate (Corning, USA) for 12 hours in media described above. Cells were treated with KAT 0, 5, 20, 40, 80, 100 for 1, 2, 4, 8 hours. In separate experiments, cells were treated with KAT 40, 60 μM for 15, 30 & 60 min. At the end of each treatment, 100 ul ATP assay reagent cell titer Glo2.0 (Promega, USA) was added in each well, shacked on a rotating shaker for 2 mins, incubated at room temperature for 10 mins in dark and luminescence was measured with BIOTEK synergy HT.

### Results:

Percent viability of Panc-2 cells treated with 5-100 μM of 3 BP analogues:

### Molecule 0

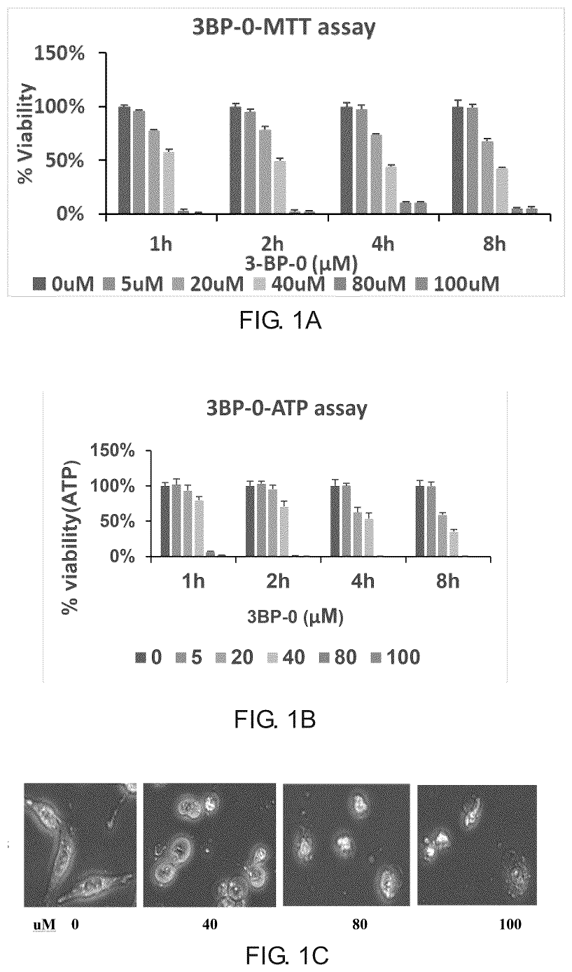

Molecule 0 is a cGMP-3-BP control. The percent viability of panc-2 cells at 40 μM was reduced after one hour and we observed 58% cell death in Panc-2 cells after 1 hour treatment, and 24% viability was observed after 8 hours. 80 μM conc. killed about 97% cells after 1 hour treatment and 100% Panc-2 cell death was observed after 8 hours. At 40 μM treatment of panc-2 cells, ATP generation was 80% at 1 hour incubation, reduced to 35% after 8 hours. 40 μM conc was sublethal in terms of MTT and ATP generation at about 24% and about 35% cell viability after 8 hours (FIGS. 1A and 1B). A change in morphology of panc-2 cells when treated at 40, 80 and 100 μM of Molecule 0 is shown in FIG. 1C.

### Molecule 1

Molecule 1 is a heavy 3-BP molecule having a structure as shown in formula II,

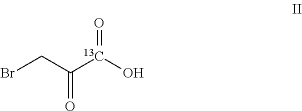

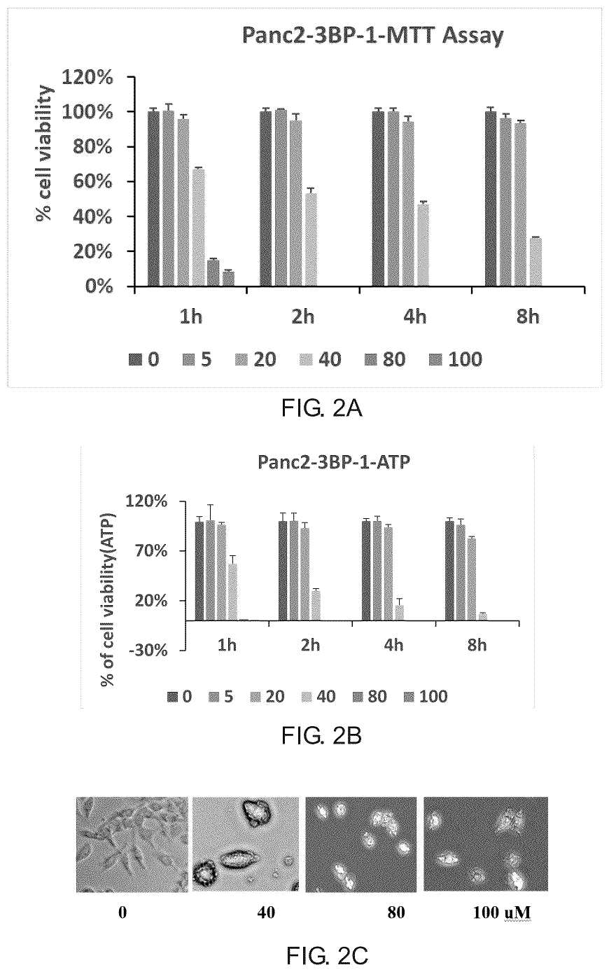

that, as is shown in FIG. 2A, at 40 μM concentration shows 54% viability in Panc-2 cells after 1 hour treatment and 30% viability after 8 hours. At 80 μM conc. killed 79% cells after 1 hour and 96% after 8 hours treatment. At 40 μM of molecule 1 treatment, ATP generation was 57% after 1 hour treatment then reduced to only 7% after 8 hours (FIG. 2B). 40 μM conc, in terms of MTT and ATP generation, showed 70% and 93% cell death after 8 hours treatment. Molecule 1 treated cells also showed rounding and membrane blabbing at 40 uM treatment after 8 hrs (FIG. 2C). About 95-97% cell death was observed when cells were treated with 80 and 100 uM conc.

### Molecule 2

Molecule 2 is a heavy 3-BP molecule having a structure as shown in formula II,

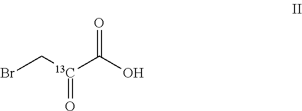

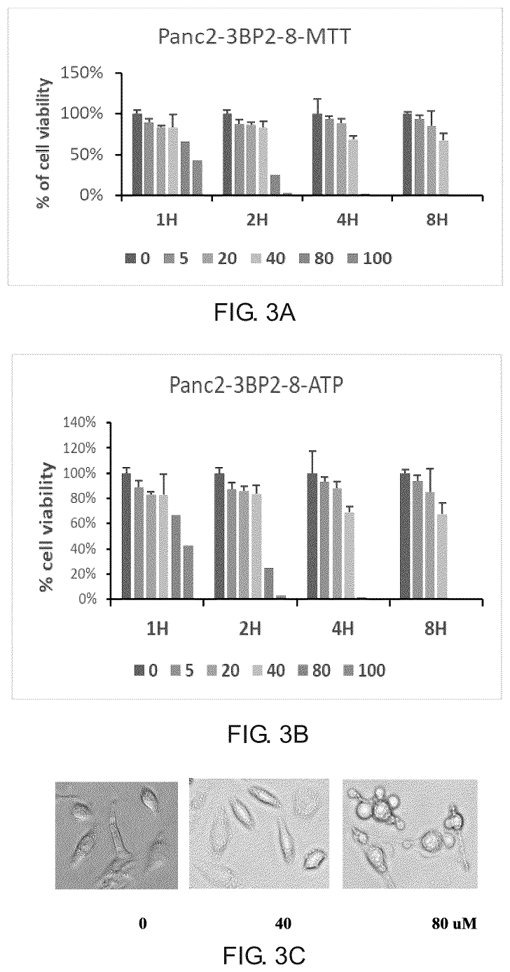

that, as is shown in FIG. 3A, treating panc-2 cells with 40 μM, we observed about 52% and about 60% viability after 1 and 8 hour treatments. At 80 μM conc., about 99% of panc-2 cells were killed after 4 hour treatment and at 100 μM about 0% viability was observed. At 40 μM of molecule 2 treatment, ATP generation was 83% after 1 hour treatment then reduced to only 68% after 8 hours (FIG. 3B). 40 μM conc. in terms of MTT and ATP generation killed 40% and 32% respectively. Molecule 2 treated cells also showed rounding and membrane blabbing at 80 μM treatment (FIG. 3C)

### Molecule 3

Molecule 3 is a heavy 3-BP molecule having a structure as shown in formula III,

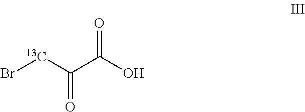

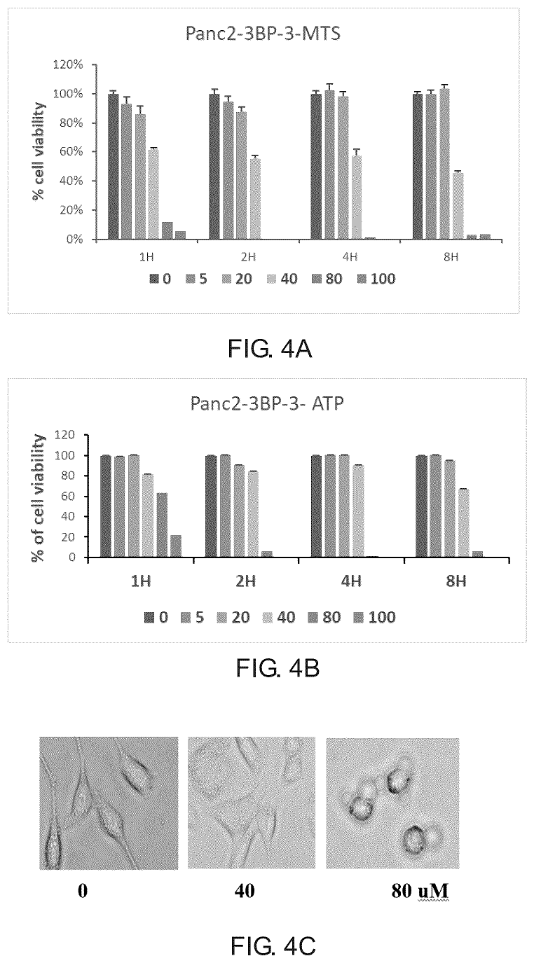

that, as is shown in FIG. 4A, treating panc-2 cells with 40 μM concentration resulted in about 62% and about 46% viability after 1 and 8-hour treatment, respectively. 80 μM conc. killed almost 88% cells after 1 hour treatment and at 100 μM only about 5% were viable after one hour treatment. At 40 μM of molecule 3 treatment, ATP generation was about 80% after 1 hour, reduced to only about 66% after 8 hours (FIG. 4B). 40 μM conc in terms of MTT and ATP generation kills about 54% and about 34% of panc-2 cells respectively. Molecule 3 treated cells also showed rounding and membrane blabbing at 40 μM treatment after 8 hours (FIG. 4C)

### Molecule 4

Molecule 4 is a heavy 3-BP molecule having a structure as shown in formula IV,

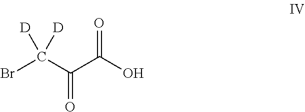

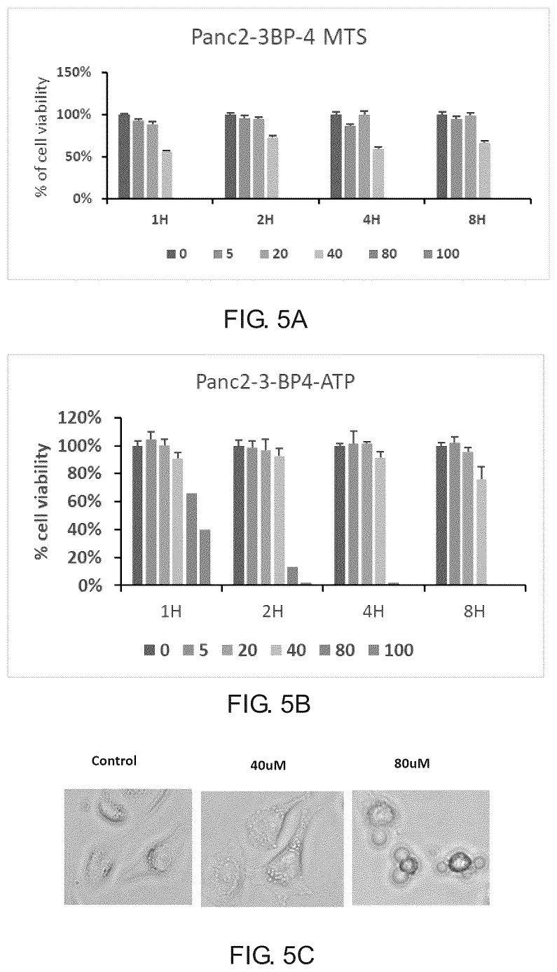

that, as is shown in FIG. 5A, treating panc-2 cells with 40 μM resulted in about 58% and about 60% viability after 1 hour and 8 hour treatment, respectively. Treatment with 80 μM conc. of molecule 4 killed about 99% of panc-2 cells after 1 hour treatment and at 100 μM about 100% cell death was observed. At 40 μM of molecule 4 treatment, ATP generation was 91% after 1 hour, reduced to about 76% after 8 hours (FIG. 5B). 40 μM conc in terms of MTT and ATP generation kill about 32-40% of panc-2 cells. Molecule 4 treated cells also showed rounding and membrane blabbing at 80 μM treatment after 2 hours (FIG. 5C).

### Molecule 5

Molecule 5 is a heavy 3-BP molecule having a structure as shown in formula V,

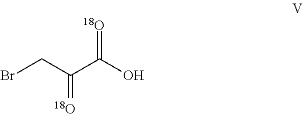

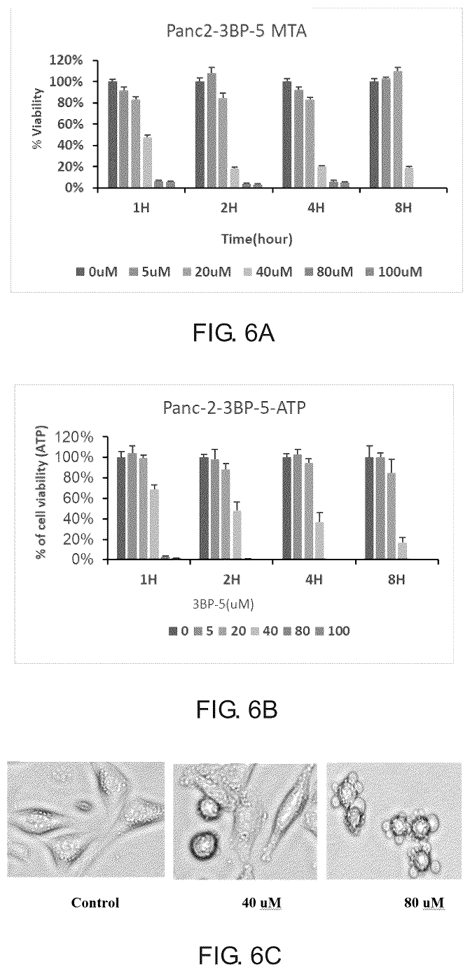

that, as is shown in FIG. 6A, treating panc-2 cells with 40 μM resulted in about 48% viability after 1 hour treatment, reduced to about 19% viability after 8-hour treatment (FIG. 6A), respectively. Treatment with 80 μM resulted in about 6% viability in panc-2 cells after 1 hour treatment, and at 100 μM about 2% viability. At 40 μM of molecule 5 treatment, ATP generation was about 69% after 1 hour treatment, reduced to about 26% after 8 hours (FIG. 6B). 40 μM conc in terms of MTT and ATP generation kills about 81% and about 74% of panc-2 cells, respectively. Molecule 5 treated cells also showed rounding and membrane blabbing at 40 μM treatment after 8 hours (FIG. 6C).

### Molecule 6

Molecule 6 is a heavy 3-BP molecule having a structure as shown in formula V,

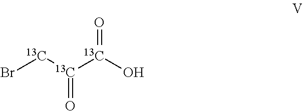

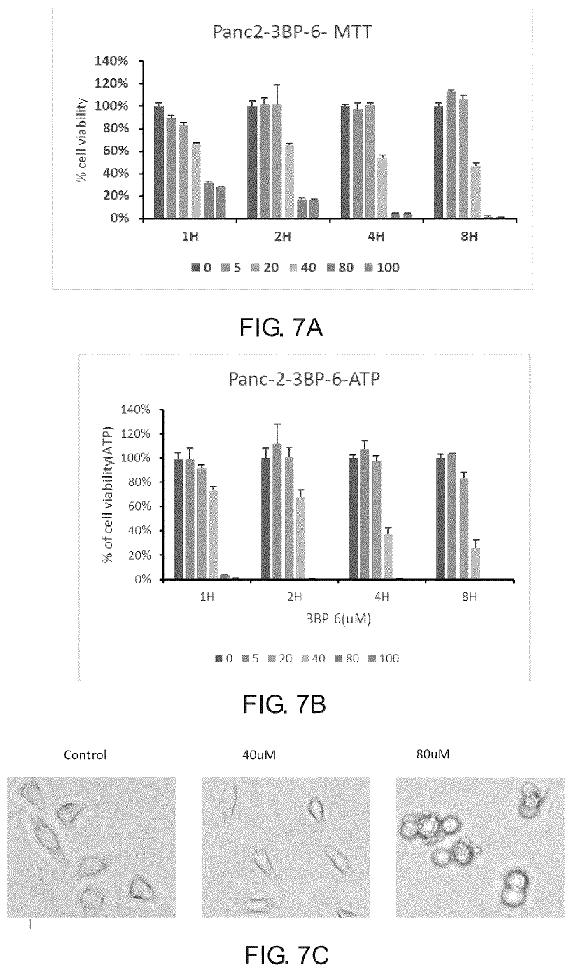

that, as is shown in FIG. 7A, treating panc-2 cells with 40 μM resulted in about 66% viability after 1 hour treatment, reduced to about 57% viability after 8-hour treatment. After 80 μM treatment for 1 hour, about 32% viability was observed and in 100 μM after 1 hour, about 28% viability was observed. At 40 μM of molecule 6 treatment. ATP generation was 73% after 1 hour, reduced to 26% after 8 hours (FIG. 7B). 40 μM conc in terms of MTT and ATP generation kills about 43% and about 74% after 8 hours. Molecule 6 treated cells also showed rounding and membrane blabbing at 40 μM treatment after 8 hours (FIG. 7C).

### Molecule 7

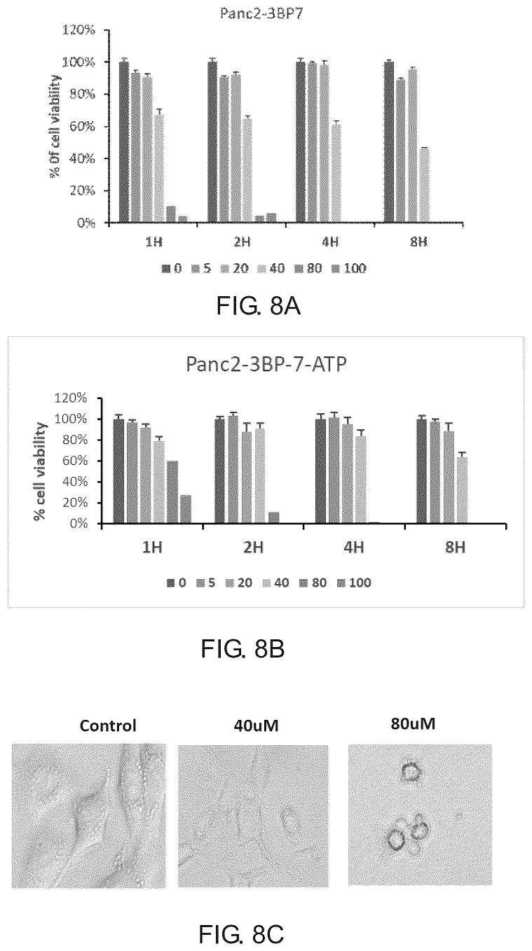

Molecule 7 was a 3-BP control. After treating panc-2 cells with 40 μM, about 67% viability was observed after 1 hour treatment, reduced to about 46% viability after 8 hours (FIG. 8A). After 80 μM treatment, about 10% viability was observed after 1 hour and about 4% viability in 100 μM was observed after 1 hour. At 40 μM of molecule 7 treatment, ATP generation was 79% after 1 hour treatment, then reduced to 64% after 8 hours (FIG. 8B). 40 μM conc in terms of MTT and ATP generation killed about 43% and about 74% after 8 hours. Molecule 7 treated cells also showed rounding and membrane blabbing at 40 μM treatment after 8 hours (FIG. 8C).

### EXAMPLES

The following examples pertain to specific embodiments and point out specific features, elements, or steps that can be used or otherwise combined in achieving such embodiments.

### Example 1. A Diagnostic Cancer Treatment Composition, Comprising

a 3-Bromopyruvate (3-BP) molecule having at least one heavy isotope according to formula I

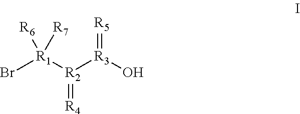

wherein R1, R2, and R3 are independently C or ¹³C;
R4 and R5 are independently O, ¹⁷O, or ¹⁸O; and
R6 and R7 are independently H or deuterium, with the proviso that at least one
R2 or R3 is ¹³C, at least one of R4 or R5 is ¹⁷O or ¹⁸O, or at least one of R6 or R7 is deuterium;
at least one sugar to stabilize the 3-BP molecule by substantially preventing the 3-BP molecule from hydrolyzing; and
a biological buffer present in an amount sufficient to at least partially deacidify the 3-BP molecule and to neutralize metabolic by-products of the 3-BP molecule.

Example 2. The diagnostic cancer treatment composition of example 1, wherein R2 is ¹³C.

Example 3. The diagnostic cancer treatment composition of example 1, wherein R3 is ¹³C.

Example 4. The diagnostic cancer treatment composition of example 1, wherein R1, R2, and R3 are ¹³C.

Example 5. The diagnostic cancer treatment composition of example 1, wherein R2 and R3 are ¹³C.

Example 6. The diagnostic cancer treatment composition of example 1, wherein R1 and R2 are ¹³C.

Example 7. The diagnostic cancer treatment composition of example 1, wherein R1 and R3 are ¹³C.

Example 8. The diagnostic cancer treatment composition of example 1, wherein R6 and R7 are deuterium.

Example 9. The diagnostic cancer treatment composition of example 1, wherein at least one of R4 or R5 is ¹⁷O.

Example 10. The diagnostic cancer treatment composition of example 1, wherein at least one of R4 or R5 is ¹⁸O.

Example 11. The 3-BP molecule of claim 1, wherein R6 is deuterium.

Example 12. The 3-BP molecule of claim 1, wherein R7 is deuterium.

Example 13. The 3-BP molecule of claim 1, wherein R4 and R5 is ¹⁷O.

Example 14. The 3-BP molecule of claim 1, wherein R4 and R5 is ¹⁸O

Example 15. The diagnostic cancer treatment composition of example 1, wherein the at least one sugar is a member selected from the group consisting of gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, and a combination thereof.

Example 16. The diagnostic cancer treatment composition of example 1, further comprising a second sugar selected from the group consisting of mannitol, erythritol, isomalt, lactitol, maltitol, sorbitol, xylitol, dulcitol, ribitol, inositol, sorbitol, and combinations thereof.

Example 17. The diagnostic cancer treatment composition of example 1, wherein the composition can include a second sugar and a third sugar independently selected from mannitol, erythritol, isomalt, lactitol, maltitol, sorbitol, xylitol, dulcitol, ribitol, inositol, sorbitol, or a combination thereof.

Example 18. The diagnostic cancer treatment composition of example 1, the composition further comprising at least one sugar selected from glycerol, inositol, and sorbitol.

Example 19. The diagnostic cancer treatment composition of example 1, wherein the biological buffer is selected from a citrate buffer, a phosphate buffer, and an acetate buffer.

Example 20. The diagnostic cancer treatment composition of example 1, wherein the biological buffer is a citrate buffer.

Example 21. The diagnostic cancer treatment composition of example 1, the composition further comprising d-lactic acid and epinephrine.

Example 22. The diagnostic cancer treatment composition of example 1, further comprising a glycolysis inhibitor.

Example 23. The diagnostic cancer treatment composition of example 1, wherein the glycolysis inhibitor is 2-deoxglucose.

Example 24. The diagnostic cancer treatment composition of example 23, wherein the 2-deoxglucose is in a concentration from about 1 mM to about 5 mM.

Example 25. The diagnostic cancer treatment composition of example 1, wherein the biological buffer is selected from a citrate buffer, a phosphate buffer, and an acetate buffer.

Example 26. The diagnostic cancer treatment composition of example 1, wherein the biological buffer is a citrate buffer.

Example 27. The diagnostic cancer treatment composition of example 1, the composition further comprising at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase: D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogenesis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, goji, mango, pomegranate, L-carnitine, selenium; a less biologically active amino acid as compared to its isomer; and mixtures thereof.

Example 28. The diagnostic cancer treatment composition of example 1, the composition further comprising a hexokinase inhibitor.

Example 29. The diagnostic cancer treatment composition of example 29, wherein the hexokinase inhibitor inhibits binding of hexokinase 1 and/or hexokinase 2 to VDAC.

1. A 3-Bromopyruvate (3-BP) molecule having at least one heavy isotope according to formula I, comprising: 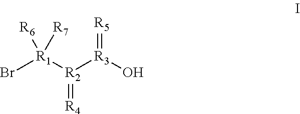
wherein R1, R2, and R3 are independently C or ¹³C;
R4 and R5 are independently O, ¹⁷O, or ¹⁸O; and
R6 and R7 are independently H or deuterium, with the proviso that at least one R2 or R3 is ¹³C, at least one of R4 or R5 is ¹⁷O or ¹⁸O, or at least one of R6 or R7 is deuterium.

2. The 3-BP molecule of claim 1, wherein R2 is ¹³C.

3. The 3-BP molecule of claim 1, wherein R3 is ¹³C.

4. The 3-BP molecule of claim 1, wherein R1, R2, and R3 are ¹³C.

5. The 3-BP molecule of claim 1, wherein R2 and R3 are ¹³C.

6. The 3-BP molecule of claim 1, wherein R1 and R2 are ¹³C.

7. The 3-BP molecule of claim 1, wherein R1 and R3 are ¹³C.

8. The 3-BP molecule of claim 1, wherein R6 and R7 are deuterium.

9. The 3-BP molecule of claim 1, wherein at least one of R4 or R5 is ¹⁷O.

10. The 3-BP molecule of claim 1, wherein at least one of R4 or R5 is ¹⁸O.

11. The 3-BP molecule of claim 1, wherein R4 or R5 is ¹⁷O.

12. The 3-BP molecule of claim 1, wherein R4 or R5 is ¹⁸O.

13. The 3-BP molecule of claim 1, wherein R6 is deuterium.

14. The 3-BP molecule of claim 1, wherein R7 is deuterium.

15. The 3-BP molecule of claim 1, wherein R4 and R5 is ¹⁷O.

16. The 3-BP molecule of claim 1, wherein R4 and R5 is ¹⁸O

17. A diagnostic composition, comprising:
a 3-Bromopyruvate (3-BP) molecule having at least one heavy isotope according to formula I 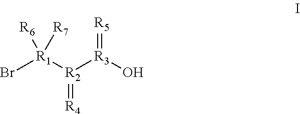
wherein R1, R2, and R3 are independently C or ¹³C;
R4 and R5 are independently O, ¹⁷O, or ¹⁸O; and
R6 and R7 are independently H or deuterium, with the proviso that at least one R2 or R3 is ¹³C, at least one of R4 or R5 is ¹⁷O or ¹⁸O, or at least one of R6 or R7 is deuterium;
at least one sugar to stabilize the 3-BP molecule by substantially preventing the 3-BP molecule from hydrolyzing; and
a biological buffer present in an amount sufficient to at least partially deacidify the 3-BP molecule and to neutralize metabolic by-products of the 3-BP molecule.

18. The diagnostic composition of claim 1, wherein R2 is ¹³C.

19. The diagnostic composition of claim 1, wherein R3 is ¹³C.

20. The diagnostic composition of claim 1, wherein R1, R2, and R3 are ¹³C.

21. The diagnostic composition of claim 1, wherein R2 and R3 are ¹³C.

22. The diagnostic composition of claim 1, wherein R1 and R2 are ¹³C.

23. The diagnostic composition of claim 1, wherein R1 and R3 are ¹³C.

24. The diagnostic composition of claim 1, wherein R6 and R7 are deuterium.

25. The diagnostic composition of claim 1, wherein at least one of R4 or R5 is 170.

26. The diagnostic composition of claim 1, wherein at least one of R4 or R5 is 180.

27. The 3-BP molecule of claim 1, wherein R6 is deuterium.

28. The 3-BP molecule of claim 1, wherein R7 is deuterium.

29. The 3-BP molecule of claim 1, wherein R4 and R5 is ¹⁷O.

30. The 3-BP molecule of claim 1, wherein R4 and R5 is ¹⁸O

31. The diagnostic composition of claim 1, wherein the at least one sugar is a member selected from the group consisting of gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, and a combination thereof.

32. The diagnostic composition of claim 1, further comprising a second sugar selected from the group consisting of mannitol, erythritol, isomalt, lactitol, maltitol, sorbitol, xylitol, dulcitol, ribitol, inositol, sorbitol, and combinations thereof.

33. The diagnostic composition of claim 1, wherein the composition can include a second sugar and a third sugar independently selected from mannitol, erythritol, isomalt, lactitol, maltitol, sorbitol, xylitol, dulcitol, ribitol, inositol, sorbitol, or a combination thereof.

34. The diagnostic composition of claim 1, the composition further comprising at least one sugar selected from glycerol, inositol, and sorbitol.

35. The diagnostic composition of claim 1, wherein the biological buffer is selected from a citrate buffer, a phosphate buffer, and an acetate buffer.

36. The diagnostic composition of claim 1, wherein the biological buffer is a citrate buffer.
